(19) United States
(12) Patent Application Publication
(10) Pub. No.: US 2001/0036626 A1
(43) Pub. Date: Nov. 1, 2001

(54) Screening assay methods and systems using target pooling

(76) Inventors: Javier A. Farinas, San Carlos, CA (US); H. Garrett Wada, Atherton, CA (US)

Correspondence Address: CALIPER TECHNOLOGIES CORP, 605 FAIRCHILD DRIVE, MOUNTAIN VIEW, CA, 94043, US

(21) Appl. No.: 09/833,080

(22) Filed: Apr. 10, 2001

Related U.S. Application Data
(60) Provisional application No. 60/197,321, filed on Apr. 14, 2000.

(51) Int. Cl. C12Q 1/00; G01N 33/53; C12M 1/34

(52) U.S. Cl. 435/4; 435/7.1; 435/287.1

(57) ABSTRACT

Methods, devices and systems for increasing the throughput of screening assays by pooling multiple target systems, which allow a library of different materials, e.g., test compounds, to be screened against the pooled targets to determine whether any of the materials affect one or more of the target systems. In preferred aspects, functioning of individual target systems is identified by differences in physical, chemical and/or optical properties particular to the target system in a target pool.

### CROSS-REFERENCE TO RELATED APPLICATIONS

This application claims priority to Provisional Patent Application No. 60/197,321, filed Apr. 14, 2000, which is incorporated herein by reference in its entirety for all purposes.

### BACKGROUND OF THE INVENTION

The pharmaceutical discovery and development is a long and extremely costly process that involves the selection of the particular disease or condition for which a treatment is sought, the generation of model systems that emulate the diseased condition, generation of large libraries of potential pharmaceutical compounds, the testing of these candidate compounds, materials or treatments against that model system, and the determination of whether promising candidate compounds will have any efficacy in the treatment of these conditions in living beings.

Because of the high costs of this overall process, a substantial amount of resources have been dedicated to the development of new and/or improved technologies with the aim toward reducing the costs and length of the various steps in the process. For example, rational drug design methods have been utilized to hypothesize about a successful drug's structure. The hypothetical drug is then synthesized and tested for pharmaceutical utility. This was theorized to reduce the amount of time required in testing large numbers of different and likely unrelated compounds. In an alternate approach, combinatorial chemistry methods have been developed in an effort to generate very diverse collections of molecules to be tested for pharmaceutical utility. The aim of this strategy was to generate as many different compounds as possible, e.g., while maintaining some or no minimal structural relationship, and screen them all for potential pharmaceutical utility. This latter approach is currently the most favored approach in pharmaceutical research.

Substantial resources have also been dedicated to the discovery of the systems that are implicated in the process of disease. The effort to sequence the human genome has contributed substantially to the number of potentially relevant target systems, e.g., those systems relevant to a particular disease or condition. With the number of potential targets and the number of potential pharmaceutical compounds increasing at such a tremendous rate, there exists a great need for high throughput pharmaceutical screening systems. A number of different groups have proposed different methods and systems for performing these high throughput assays. Conventional methods have employed large numbers of multiwell assay plates and complicated systems of robots to handle reagent addition and assay reading.

More technically advanced methods and systems have also been proposed. For example, U.S. Pat. No. 5,942,443 describes a microfluidic approach to high throughput pharmaceutical screening where one or more components of a target system are flowed through a microfluidic channel, while the different candidate compounds are introduced into the channel. Effects of the candidates on the model system are then detected within the channel. By performing these assays at the microscale, one gains advantages in terms of the quantity of reagents used, the speed at which a particular individual assay is carried out, and the number of parallel assays that can be carried out.

Despite these developments, there still exists a need to expand the rate at which one can screen increasing numbers of potential pharmaceutical compounds for effects on increasing numbers of pharmaceutical targets. The present invention meets these and a variety of other needs.

### SUMMARY OF THE INVENTION

The present invention is generally directed to methods, devices and systems for increasing the throughput of screening assays by pooling multiple target systems. The method allows a library of different materials, e.g., test compounds, to be screened against the pooled targets to determine whether any of the materials affect one or more of the target systems. In preferred aspects, functioning of individual target systems is identified by differences in physical, chemical and/or optical properties.

The present invention provides a method of performing a screening assay. The method comprises providing a first target mixture in a first reaction vessel. The first target mixture comprises at least first and second different target systems. At least one test agent is introduced into the target mixture and the effect of the test agent on the first and second target systems is determined.

A further aspect of the present invention is a system for performing high throughput screening assays. The system comprises a reaction vessel containing a first target mixture. The first target mixture comprises at least first and second target systems, the first target mixture being different from the first target system. A test agent sampler is also included for sampling a test agent and introducing the test agent into the reaction vessel. The system also includes a detector positioned in sensory communication with the first target mixture. The detector is configured to detect an effect of a test agent on the first and second target systems.

### BRIEF DESCRIPTION OF THE FIGURES

FIG. 1 schematically illustrates an overall system for carrying out the screening methods of the present invention.

FIGS. 2A and 2B schematically illustrates two different microfluidic devices, having different channel layouts for carrying out variations of screening assay methods of the invention. FIG. 2C illustrates either microfluidic device from a side perspective.

FIG. 3A and 3B are plots of data from two target systems separately maintained and monitored during a screening assay.

FIG. 4 is a plot of the same two target systems shown in FIG. 3, except that the systems are pooled in a single reaction vessel and monitored simultaneously.

FIG. 5 shows a channel layout of a microfluidic device used in carrying out methods of the present invention.

FIG. 6 shows data plots from the screening of two pooled cell-based target systems. FIG. 6A and 6B illustrates plots of the fluorescent response of the same pooled cell lines to increasing carbachol concentrations. FIG. 6C and 6D show the fluorescently indicated response of pooled CHO-M1 cells and THP-1 cells to increasing concentrations of UTP.

### DETAILED DESCRIPTION OF THE INVENTION

I. Introduction

The present invention generally provides methods, devices, kits and systems for use in screening assay operations. As used herein, the term “screening” refers to the testing of relatively large numbers of different agents, referred to herein as “test agents” against a target system, for potential effects on that target system. The relatively large numbers of agents generally include more than about 50, typically more than about 100, preferably, more than 1000, and upwards of 1,000,000 or more different test agents or materials. Typically, these screening assay operations are used in screening potential pharmaceutical candidates or test compounds for effects on target systems. While this is generally the focus of discussion of the methods and systems described herein, it will be appreciated that other screening assays, e.g., toxicology screening assays, functional genomics assays, and the like are equally used in conjunction with the methods and systems of the invention. The methods and the systems of the invention take advantage of “target pooling” which involves providing a single mixture that includes more than one target system. Unlike methods of pooling potential pharmaceutical compounds for enhancing throughput, target pooling methods do not suffer from potential cross-over effects between the pooled targets. In particular, in pooling candidate compounds, one runs the risk that two or more of the pooled compounds may alter the effect that one compound by itself would have. This could be a synergistic effect when combined in the mixture, or could be a reduction or elimination of an effect, thereby causing one to bypass a potentially useful compound.

In general, pooled targets are placed into a reaction vessel, and the pooled target mixtures are separately screened against large numbers, or “libraries,” of different compounds, also referred to herein as “test agents” or “test compounds.” These test agents or compounds can be any of a variety of different materials or mixtures of materials. For example, in pharmaceutical screening operations, test compounds are generally small molecule, drug-like compounds, peptides or proteins, including proteins and/or peptides presented or expressed on cell surfaces, phage display libraries, or the like. However, in these and other screening applications, test compounds can include macromolecular assemblies or complexes, extracts of plant, fungal, animal, bacterial, or other materials. Test compounds may exist in solution or they may be coupled to particles, e.g., beads or cells, for the screening operation.

In pharmaceutical screening operations, entire libraries or substantial portions thereof are typically screened against large numbers of different target systems. The effect, if any, of a particular test compound on any one of the pooled targets is then detected. Different targets within a pool optionally are detectable by the same methods and properties or have different bases for detection. In the case where a single detection scheme, e.g., a single wavelength fluorescence, is used to monitor each of the pooled target systems, positive results cannot be readily attributed to a single target system within a target pool. Accordingly, in such cases, it may be necessary to individually screen a promising candidate against each target system in a given pool. Typically, a lack of specificity in this regard is not problematic, as the frequency of promising candidates in a particular library will typically be relatively low. In alternative preferred aspects, differential detection strategies are employed for each of the target systems in a given pool, thereby allowing attribution of an effect of a promising compound to a particular target system.

While the use of pooled targets does not necessarily increase the rate at which an individual target screen takes place, it does increase the overall throughput of a screening facility by allowing the screening facility to multiplex different screens in a single screening process. In particular, by pooling targets, one can increase the overall throughput of a screening facility or operation by a factor equivalent or substantially equivalent to the number of pooled targets. In accordance with the present invention, targets may be pooled as liquid mixtures, e.g., as mixtures of liquid reagents, as particulate compositions, e.g., where components or reagents of the target system are tethered to solid supports, e.g., beads, or as cell suspensions, where the cells contain the target systems. In particular, cell suspensions may include a cell group that contains two or more targets, e.g., expressed by the cells of the cell group or multiple different cell groups, where each group contains only a single target system.

II. Pooled Targets

A. Target Systems

Target systems typically include one or more components of any biological and/or biochemical system for which an agent that modulates activity of that system could be useful. For example, a system that is identified as being implicated in the pathology of a particular disease or condition may be screened in order to identify agents that affect that system's involvement in the pathology. Generally, such target systems can be screened in order to identify lead pharmaceutical compounds, or in an effort to identify ligands for orphan receptor systems, or the like. A few examples of particularly interesting biochemical systems include receptor-ligand systems, signal transduction systems, ion channel or pump systems, enzyme-substrate interactions, specific binding interactions, e.g., nucleic acid interactions with other nucleic acids or proteins, protein-protein interactions antibody-antigen systems, and the like. From these relevant biochemical systems, one or more particular components may be identified as serving a critical or important function within the system, which function is initiated or altered in the case of a particular pathology or condition. In some cases, a component of a biochemical system that has no identified function is used as a target, in order to facilitate identification of pharmacologically relevant target systems. The one or more component is then identified as a “target” against which libraries of compounds may be screened to determine whether those compounds have any effect on the target, e.g., its function, its interaction with other components of the target system, or events that are initiated by the action or function of the target.

1. Receptor Target System

As noted above, one example of a target system is a receptor or receptor-ligand system. In particular, the interaction of a receptor with its ligand, an alteration in that interaction, and/or the downstream events that follow that interaction can be important events in a particular pathology. As such, screening assays often use model receptor-ligand systems as screening targets (also referred to herein as “target systems”). Some examples of often used receptor target systems include G-protein coupled receptor (“GPCR”) systems. In particular, these receptor systems are generally implicated in a wide variety of different pathologies, including cardiovascular, neurological, immunological, digestive and other pathologies. Other classes of generally useful receptor target systems include nuclear hormone receptors, ligand gated ion channels and protein kinase receptors.

In general, receptor target systems typically comprise at least two components of a biochemical system, namely a selected receptor and the ligand or agonist to that receptor. However, in some cases, one is screening for agonists or ligands to a given receptor. In such cases, the receptor target system may simply comprise the receptor portion of the system, as well as an appropriate reporter mechanism. The receptor in a given target system may be present as an aqueous or soluble preparation. However, in preferred aspects, the receptor component of the system is included as a portion of a whole cell in a suspension of viable whole cells, e.g., as a cell surface receptor or internal receptor. Receptors may be native to the particular cell line that is being used, or the cell line may be engineered to express a desired receptor, whereby the cell functions as a carrier and/or reporter system for the receptor.

Reporter systems typically couple ligand binding or activation of a receptor associated with a given cell, to the ultimate expression by the cell of a detectable event, i.e., production of a detectable protein, e.g., β-galactosidase, etc., or other material, change in some physical characteristic, or the like. Engineering of receptor linked enzyme systems has been practiced by those of ordinary skill in the art, and is generally described in, e.g., Methods for Cloning and Analysis of Eukaryotic Genes, Bothwell, Yamacopoulos and Alt (Jones & Bartlett, Boston Mass.).

In the case of many receptor target systems, the natural action or function of the receptor can be used to monitor the target system. For example, in target systems that utilize GPCRs, changes in ion flux of the cells can be used to monitor changes in receptor activity in response to that receptor's ligand. Typically, changes in ion flux are readily monitored using intracellular indicator dyes that are specific for different ionic species, e.g., Calcium, Sodium, protons, etc. Such dyes are typically commercially available from, e.g., Molecular Probes, Inc. (Eugene, Oreg.), and include, e.g., commonly used calcium indicators include analogs of BAPTA (1,2-bis(2-aminophenoxy)ethane-N,N,N′,N′-tetraacetic acid), such as Fura-2, Fluo-2 and Indo-1, which produce shifts in the fluorescent excitation or emission maxima upon binding calcium, and Fluo-3 and Calcium Green-2, which produce increases in fluorescence intensity upon binding calcium. See also, U.S. Pat. No. 5,516,911. Sodium and potassium sensitive dyes include SBFI and PBFI, respectively (also commercially available from Molecular Probes). Examples of commercially available chloride sensitive indicators include 6-methoxy-N-(sulfopropyl)quinolinium (SPQ), N-(sulfopropyl)acridinium (SPA), N-(6-methoxyquinolyl)acetic acid, and N-(6-methoxyquinolyl)acetoethyl ester (Molecular Probes, Inc.), all of which are generally quenched in the presence of chloride ions. Changes in the level of fluorescence are then attributable to changes in ion flux caused by the receptor activity.

In alternative arrangements, interactions between receptors and ligands are monitored using methods that indicate the binding of the two components, or by binding of the receptor to a binding partner, e.g., by measuring changes in the level of depolarized fluorescence emitted by the target system. In particular, one of the receptor, ligand or binding partner is provided with a fluorescent label. This labeled component, when in a non-complexed form, e.g., a ligand not bound by its receptor, emits a particular level of depolarized fluorescence when excited using a polarized light source, due to the rotational diffusion of the relatively small labeled component. Changes in the size of the labeled component, e.g., resulting from binding of a labeled ligand by its receptor, reduce the rotational diffusion of the labeled group (now the complex), resulting in a reduction in the level of emitted depolarized fluorescence. This level of depolarized fluorescence provides a quantitative measurement of the level of interaction between the two species. The monitoring process is then carried out as test compounds are introduced into the target system, so that any effects of the compound on the interaction between the receptor and ligand can be determined. Alternatively, changes in the sizes of the labeled component can be measured by fluorescence correlation spectroscopy.

As noted above, a wide variety of different receptor systems can be screened as target systems in the methods of the invention provided that the receptor function, or changes in that function are detectable. These include, by way of example, GPCRs, tyrosine kinase receptors, cytokine receptors, adhesion factor receptors, antigen receptors (e.g., surface immunoglobulin), T-cell receptors, ion channel receptors and the like.

2. Enzyme Target Systems

A variety of enzymes are also used as target systems in pharmaceutical research. For example, kinase and phosphatase enzymes are of particular interest due to their activity in critical cell signaling cascades, e.g., through phosphorylation and dephosphorylation of downstream proteins and messenger compounds, which are implicated in a number of important pathological events. Proteases also are routinely screened in pharmaceutical research, due to their roles in immune system evasion, blood coagulation, protein turnover, and a variety of other pathology associated events. Other enzyme classes, e.g., carbohydrases (e.g., amylases, glucanases, etc.), nucleases, etc.

Enzyme target systems typically include a substrate for the enzyme target. Although natural substrates can be used, it is typically desirable to use a model substrate for which the enzyme has a high affinity. More preferred still are substrates that will ultimately facilitate detection or monitoring of the function of the enzyme. For example, fluorogenic substrates are most preferred for their ease of use. Such substrates typically have a particular fluorescent profile, e.g., high or low fluorescence, or fluorescent emission or excitation at a particular wavelength. When acted upon by the enzyme of interest, however, the product will have a detectably different fluorescent profile, e.g., a lower or higher fluorescence or a shift in the excitation or emission spectrum. In most cases, fluorogenic substrates are non-fluorescent or have a relatively low level of fluorescence at a given wavelength, but produce a product that has a substantially higher fluorescence at the same wavelength, when acted upon by the enzyme of interest. In general, fluorogenic substrates for the more important classes of enzymes are commercially available from, e.g., Bachem or Molecular Probes, Inc. For example, Fluorosceindiphosphate and diFMUP are examples of commercially available phosphatase substrates. Similarly, BOC-Fluoresceinated peptides, e.g., Boc-Fluoroscein-SRAMC and ZGSRAMC are generally useful as fluorogenic protease substrates.

Non-fluorogenic substrates are also useful in the methods of the present invention. In particular, in some cases fluorogenic substrates may not be readily available for a given enzyme activity. For example, fluorogenic substrates are not widely available for kinase enzymes, e.g., where a phosphorylated product has a distinctly different level of fluorescence than the substrate. Instead, however, such products do possess a substantially different level of charge. The difference in charge is then detectable either using a mobility shift/electrophoretic separation detection method, e.g., separating substrate and product for quantitation. Examples of non-fluorogenic substrates include fluorescently labeled phosphorylatable peptides for kinases, that are generally readily synthesized or can be commercially obtained through, e.g., SynPep, Inc., and fluorescent peptide substrates for proteases, generally available through the same sources.

Alternatively, methods have been described for assaying such changes in molecular charge by adding relatively large (as compared to the phosphorylated product) polyionic species, e.g., polylysine, polyarginine, or the like, and detecting the resulting complexation by changes fluorescence polarization. Thus, where a product is produced having greater or less charge, it will bind to a different extent to the added polyion, yielding differential changes in fluorescence depolarization. This method is described in substantial detail in commonly assigned, copending International Patent Application No. 00/72016, which is incorporated herein by reference in its entirety for all purposes.

In operation, two or more enzymes are provided in a single pooled target mixture. The enzymes are then combined with their respective substrates, which are also typically pooled. The base level of enzyme activity is then measured. This assay is then repeated in the presence of individual test compounds, and the level of enzyme activity on the substrates is monitored. Where a deviation is seen in the enzyme activity in the presence of the test compound versus the absence of a test compound, it is indicative that the test compound has an effect on one or more of the pooled enzyme/substrate systems. Typically, the test compounds are introduced into the enzyme pool prior to the addition of the pooled substrates, or in the substrate pool prior to their addition to the pooled enzymes.

3. Nucleic Acid Systems

Nucleic acids and their interactions with other biochemical species are also often examined as target systems in pharmaceutical screening operations. For example, in many instances it is desirable to be able to ascertain, in a high throughput format, whether potential pharmaceutical candidates have effects on the interactions between nucleic acids and nucleic acid binding proteins. Such interactions are often critical in cellular activation pathways leading to increased or decreased expression of particular genes. Typically, such target systems comprise a nucleic acid sequence that includes a recognition sequence for the nucleic acid binding protein that is to be screened. The nucleic acids are generally provided within the target pool as short probes that are fewer than 200 nucleotides in length, preferably fewer than 50 nucleotides in length, and more preferably, fewer than about 30 or even 20 nucleotides in length. The target system also typically includes that protein that recognizes and binds to a portion or multiple portions of the nucleic acid probe. Again, as noted above, the nucleic acid probes and binding proteins may be provided free in solution, or they may be introduced into or exist within a cell suspension. Performance of this type of screening assay is described in International Patent Application No. PCT/US00/35657, which is hereby incorporated herein by reference in its entirety for all purposes.

Detection of binding of nucleic acids to other species is typically accomplished using the methods described with respect to non-fluorogenic assays, and preferably using fluorescence polarization based detection, where the nucleic acid probe bears the fluorescent group, or as a change in the electrophoretic mobility of the complex versus the free labeled component, e.g., nucleic acid probe.

4. Ion Channel Systems

Ion channels represent another class of target systems that are screened against using the methods and systems of the present invention. Ion channels are important in regulating the transmembrane potential of cells and cellular organelles and play a critical role in electrical signaling processes in the nervous system. Changes in ion channel activity are typically controlled by: binding of ligands to the ion channel; post-translational modifications of the channel; changes in transmembrane potential; or mechanical stimulation. Because of the importance of these systems in a variety of biological systems, screens are often carried out to identify agents, which are capable of affecting the normal function of these channels.

Typically, ion channel target systems comprise one or more subunits of the ion channel together with a cell membrane, organelle membrane, cellular membrane fragment, artificial membrane or lipid micelles within which the ion channel resides. In preferred aspects, the ion channel to be screened is expressed as part of a viable cell's plasma membrane. In such cases, ion channel targets may be native to the cell line that is being used or may be heterologously expressed in a host cell line. The activity of ion channel systems is typically measured by detecting changes in the flux of ions across the membrane, by detecting changes in transmembrane potential, or by detecting downstream events that flow from the function of the ion channel, e.g., reporter gene activation, etc. Changes in ion fluxes or downstream events can be measured generally in the same fashion as described for receptor systems, above.

In the case of transmembrane potential measurements, several methods are available for determining the changes in transmembrane potential which are directly applicable in the methods and systems described herein. For example, Tsien et al. (U.S. Pat. No. 5,661,035) describes a method for optical detection of transmembrane potential by measuring changes in the FRET between a translocating fluorescent anion and a fluorophore distributed asymetrically adjacent to the membrane, which changes result from changes in transmembrane potential. Alternatively, International Patent Application No. PCT/US00/27659, which is incorporated herein by reference for all purposes, describes methods for determining transmembrane potential changes by measuring the rate of uptake of membrane permeable fluorescent ions, which changes depending upon the transmembrane potential.

5. Others

A long list of pharmaceutically relevant target systems are known to those of ordinary skill in the art and generally span the full range of biochemical activities outlined in U.S. Pat. No. 5,942,443, which is incorporated herein by reference in its entirety for all purposes. In general, such systems include, e.g., G-protein coupled receptors, (both membrane and nuclear), ion channels, transporters, pumps, catabolic and anabolic enzyme systems (e.g., proteases, phosphatases, kinases, etc.) binding partners (protein-protein, nucleic acids, nucleic acid-protein), and the like. In short, virtually any detectable enzymatic, signaling, transport or binding event can be used as a target system in accordance with the methods described herein. Typically, such target system types are readily applicable to the systems described herein.

B. Target Pools

1. Generally

In accordance with the present invention, at least first and second target systems are combined into a first target mixture. In a first aspect, a target mixture is comprised of a mixture of the components that make up the first and second target systems, wherein at least one of the target systems is present in liquid form, e.g. as a solution of the components of the target system. Typically, in such cases, all of the pooled target systems will be present in the same solution form. By way of example, in a solution based target mixture that comprises at least two enzyme target systems, the target mixture typically includes a solution of first and second enzymes that are components of the first and second target systems. Appropriate substrates for the first and second target systems are often included in a target mixture, although in some cases, substrates are added at a later point in the screening operation, e.g., after a test compound is introduced into the target mixture.

In another aspect, a target mixture is comprised of at least one target system that is associated with particles in a suspension. Such particles include, e.g., bead based suspensions, cellular suspensions and the like. In the case of bead based target systems, at least one component of the target system is typically immobilized on a flowable solid support, e.g., agarose, cellulose, dextran, acrylamide, or silica beads. In certain preferred aspects, the target system includes a cell suspension, where the target systems are embodied, at least in part, in the cells of the cell suspension. Target systems may be natively associated with the cells in the cell suspension, e.g., where the cells naturally express the receptor, enzyme, nucleic acid, or other component of a biochemical system of interest. Optionally, the target systems are engineered to express the component(s) of the target system, or have the component(s) of the target system exogenously introduced into the cells prior to the performance of the screening assay. Again, other components of the target system may be present within the cell suspension or are alternatively introduced at a later point, e.g., after a test compound is introduced into the target mixture.

The cell suspensions described herein typically comprise one or more different cell groups, where each different cell group comprises one or more different target systems. For example, in some cases, a cell suspension includes at least two different cell groups, where one cell group comprises a first target system, e.g., the cells express a first receptor and associated elements, i.e., reporter system. A second cell group within the cell suspension expresses a second target system, e.g., another receptor and reporter system. The two cell groups are then pooled in the same suspension. In some cases, at least one target system, e.g., a first cell group, is a reference cell line, whereas the second cell group is the same as the first, except that it has been engineered to express a particular target system, e.g., a cell surface receptor. In such cases, the reference cell line functions as a control target system, e.g., where the normal level of function in the reference cell line is the particular target system. For example, a host cell line may be transfected with a GPCR, the nontransfected host cell line functions as the first cell group, while the second cell line functions as the second group. The two cell lines are then pooled for the screen. The normal function of the host cell group represents the first target system, while the transfected cell line represents a second target system in the target pool.

Alternatively, or additionally, a single cell group within the suspension may comprise more than one different target system, e.g., expressing more than one receptor/reporter system of interest. In this latter case, the overall suspension may be made up entirely of a single, multiple target system claim group. However, to further increase the number of targets, multiple claim groups that each comprise multiple target systems may be combined in a single cell suspension.

In certain preferred aspects, a particular target mixture will comprise related target system types, e.g., receptor/reporter systems, enzyme systems, etc., in order to allow for optimization of overall conditions for the various screening assays that are taking place. For example, in a particular cell suspension, the multiple target systems are typically comprised of a plurality of different receptor systems, e.g., where the cells express multiple different receptor/reporter systems, or multiple different cell groups each expressing at least one different receptor/reporter system. As many receptor systems are monitored using similar or identical properties, e.g., reporter functions, changes in ion flux, etc., it is often desirable to provide an overall environment for the target mixture that is optimized, for all target systems that are present, optimization that is facilitated by the relatedness of the various target systems.

2. Exemplary Pooled Target Systems

The pooled target systems described herein are generally useful in all of the earlier described examples of pharmaceutically useful target systems, e.g., receptor target systems, enzyme target systems, nucleic acid target systems, etc.

By way of example, pooled receptor target systems typically include multiple receptors, either free in solution, or associated with one or more groups of cells in a suspension of cells. The target pool also typically includes the ligands for the various receptors. These are typically introduced to the assay system as a pool of the various ligands to the various receptors, which introduction can be prior to or after addition of the test compound or compounds that are to be screened, as described in greater detail below. Once the components of the target pool are combined, e.g., the receptor and ligand, the interaction between those components is monitored. In the case where test compounds are introduced into the pooled target systems, any effect of that test compound on one or more of the target systems in the pool is measured as a difference from the interaction in the absence of the test compound. In the case of pooled enzyme target systems, it is generally preferred to provide the enzymes and substrates free in solution, as opposed to associated with cells, in order to provide optimal availability of the two components for each other. Similarly, nucleic acid based target pools are also optionally provided free in solution or may be provided disposed within cellular suspensions, e.g., as described in PCT/US00/27659, and Published International Patent Application No. WO 99/67639, each of which is incorporated herein by reference in its entirety for all purposes.

III. Screening Assay Methods

In the methods of the present invention, target pools are provided and used in screening test compounds for a potential effect on the various target systems present therein. As noted above, these target pools may comprise solution based reagents, reagents associated with beads, or they may be cell based, in whole or in part. The target systems used in these methods have a detectable signal that is associated with the function or operation of that system, e.g., a detectable product, detectable interaction, or the like.

Detectable signals are optionally optically detectable signals, chemically detectable signals, electrochemically detectable signals, physically detectable signals, or the like. In particularly preferred aspects, optically detectable signals are used to monitor the function of a particular target system. Fluorescent, chemiluminescent and chromic signals are particularly preferred examples of optical signals, with fluorescent signals being most preferred. Typically, fluorescent signals may be based upon a fluorogenic operation of the target system (e.g., where the operation results in the creation of a fluorescent species where no such species existed prior to the operation), or the operation of the target system to change the properties of an existing fluorescent species (e.g., changing the molecular charge of the species, or changing its rotational diffusion rate). In the latter case, detection of the optically detectable species is then carried out by distinguishing substrate from product based upon charge, e.g., through electrophoresis (see, e.g., U.S. Pat. No. 5,942,443), or by using fluorescence polarization detection methods (see, e.g., WO 00/72016).

Due to its simplicity, fluorogenic target systems are most preferred. In particular a variety of different substrates and or dyes are available that produce a distinguishable fluorescent signal when they are acted upon by a particular target system. For example, a variety of different fluorogenic substrates are available for different enzyme systems, where action of the enzyme on the substrate produces a fluorescent product where the substrate either was not fluorescent or had a fluorescence spectrum distinguishably different from the product. Similarly, a variety of dyes are available that emit a particular fluorescent signal based upon the environment in which they are disposed. For example, in the case of cell based assay systems, a variety of dyes are available that are incorporated into the cells and which produce a fluorescent signal based upon the relative presence or absence of particular ions within the cell. A variety of different intracellular ion specific dyes are generally commercially available from Molecular Probes, Inc. (Eugene, Oreg.). Although these systems which exhibit different fluorescence depending upon their environment are not generally uniformly referred to as “fluorogenic,” for the purposes of the instant disclosure, the term fluorogenic specifically encompasses these and similar systems.

In particularly preferred aspects, the target systems in a pooled target mixture will comprise different signaling operations. For example, a first target system will produce a fluorescent signal within a first set of wavelengths, while another target system in the pooled target mixture will produce a fluorescent signal having a different set of wavelengths. By using independently detectable signals or “readouts,” e.g., distinguishable signals, for each of the target systems or subsets of the target systems in the overall pooled target mixture, one can monitor the various different target systems independently, and thus distinguish their contribution to the overall signal profile. This independent monitoring has at least a two-fold advantage over detecting all target systems at the same wavelength. First, by differentiating the target systems, one can ascertain immediately which target system is affected by a given test compound, as opposed to re-screening each target system individually when some effect is observed. Additionally, independent detection allows one to maintain a low signal/noise ratio for each target system, allowing easier identification of alterations to the signal based upon a particular test compound. This is in contrast to a single detection scheme, where the effects of a test compound on one target system are diluted out, in terms of the signal to noise ratio, by the lack of effects on any of the remaining target systems. Different signaling operations can also be obtained by using probes with different electrophoretic mobilities. In particularly preferred aspects, target systems comprising one or more cell groups are distinguished by labeling each cell group with different fluorescent spectra (shape or intensity), e.g., one or more different fluorescent labels per cell group and cell fluorescence is read on a cell by cell basis.

The target systems are screened against test compounds by mixing the test compounds with the pooled target mixtures, and detecting an effect on the amount of the detectable signal that is produced by the system. This signal is then compared to the signal produced by the system in the absence of the test compound (“the control signal”). As noted, the signal is preferably detected for each of the different target systems in the pooled target mixture by virtue of the distinguishable signal from each target system in the pool. A deviation of the target signal over the control signal is indicative that the particular test compound has an effect on the particular target system. In alternative methods, the overall signal is measured and compared to the control signal level. Where a deviation occurs, it is indicative that at least one of the target systems in the pool is affected by the test compound. Each of the target systems may then be independently interrogated against the test compound to identify the target system affected.

In many cases, the test compound may be added to a portion of the target system prior to the addition of another component of the target system. For example, in the case of enzyme target systems, it is often desirable to incubate the enzyme of interest with the test compound prior to the introduction of the requisite substrate for that enzyme.

Large numbers of different test compounds are screened by combining them with separate volumes of the pooled target mixture. This can generally be carried out in a large number of separate, discrete reaction vessels, e.g., wells in a multiwell plate, i.e., 96, 384 or 1536 well plates, or separate channel networks in a capillary device or system or microfluidic channel network. Alternatively, and preferably, separate screening assays are carried out within microfluidic channels, where separate test compounds are serially introduced into and screened against a continuous stream of the pooled target mixture.

IV. Systems

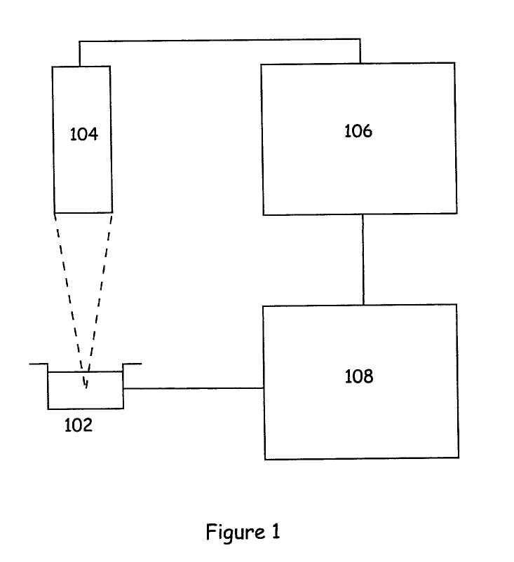

A wide range of assay systems can be used in practicing the methods described herein. For example, conventional screening assay systems that employ, e.g., test tubes or multiwell plates can be used in the methods described herein by simply providing pooled target systems within the reaction tubes or wells. Similarly, microfluidic devices and systems are also readily employed in high throughput screening assays using the pooled target methods described herein. The systems of the invention typically employ either conventional or microfluidic devices, e.g., reaction receptacles, in addition to detection instrumentation, and control instrumentation for the control of the other instruments and devices, as well as for gathering and storing data, analyzing that data, and the like. FIG. 1 schematically illustrates an overall assay system in accordance with the present invention.

As shown, the overall system 100 includes a reaction vessel 102, and a detector 104 that is in sensory communication with the contents of the reaction vessel 102 (as indicated by the dashed lines). The detector is operably coupled to a processor or computer 106 that receives, stores and optionally analyzes the data that is generated by the detector regarding the contents of the reaction vessel 102. An optional controller 108 is also provided. The controller is typically operably coupled to the processor or computer, which instructs the operation of the controller in response to user programmed instruction sets. Such controllers can include controllers that control the position of the reaction vessel, e.g., robotic controllers for plate handling robots, and the like. Alternatively or additionally, the controller comprises a flow controller, e.g., where the reaction vessel is a flow through vessel, i.e., a microfluidic channel or channel network. In either event, the controller is typically operably coupled to the reaction vessel, e.g., mechanically in the case of robotic controllers, or electrically, pneumatically or fluidically, in the case of flow controllers.

A. Conventional Assay Systems

As noted above, the target pooling methods are highly useful in conventional assay formats, where assay reagents are added into a reaction mixture in a particular reaction vessel, e.g., a well in a multiwell plate, a test tube, or the like, e.g., as shown in FIG. 1. In particular, the pooled target mixture is generally added to the wells of a multiwell plate. Additional reagents are then added to the wells of the plate, e.g., test compounds, and additional components of the target systems, e.g., ligands for pooled receptors, substrates for pooled enzymes, and the like. In the case of high throughput screening methods, different test compounds are added to each well of the plate or collection of plates, and individual affect on the pooled target system is determined as compared to a control, e.g., where no test compound or a known effector compound (agonist or antagonist) for the pooled target system is added. In some cases, it may be desirable to assay a positive control for each of the pooled target system, and/or a positive control that has an effect on all of the polled target systems.

Where a reaction mixture yields a result that is different from a negative control or approaches a result of a positive control, it is indicative that the test compound added in that well is an effector of at least one of the target systems in the pooled target system. As referenced previously, in the case where each target system produces a signal that is distinguishable from the other target systems, positive screening results are easily attributed to the appropriate target system. In the case where only a single detectable signal is used for the overall system, positive screening results require the user to identify the test compound responsible, and go back in a secondary screen to identify the specific target system affected. This is generally done in a separate reaction vessel, e.g., a multiwell plate

B. Microfluidic Assay Systems

Microfluidic assay systems are also useful in the target pooling screening methods described herein. In general, the microfluidic device or channel functions as the reaction vessel, as described above, e.g., as the vessel 102 of FIG. 1. Specifically, the pooled target mixture is introduced in a microfluidic channel in a microfluidic device, where additional reagents are brought in and added to the target pool, including test compounds, additional reagents and components of the target system, etc. Microfluidic systems provide numerous advantages over conventional systems, in that they utilize far smaller amounts of reagents, including target system reagents. Further, their small scale and integrated structure permit multiple operations, e.g., reagent additions, separations, etc., to be performed in a single integrated channel network.

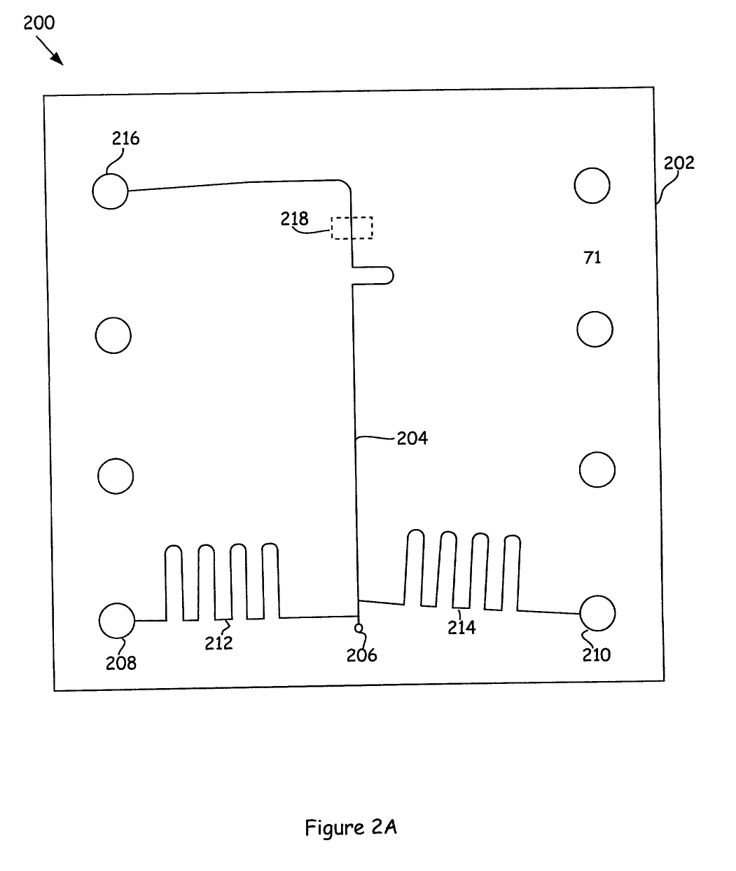

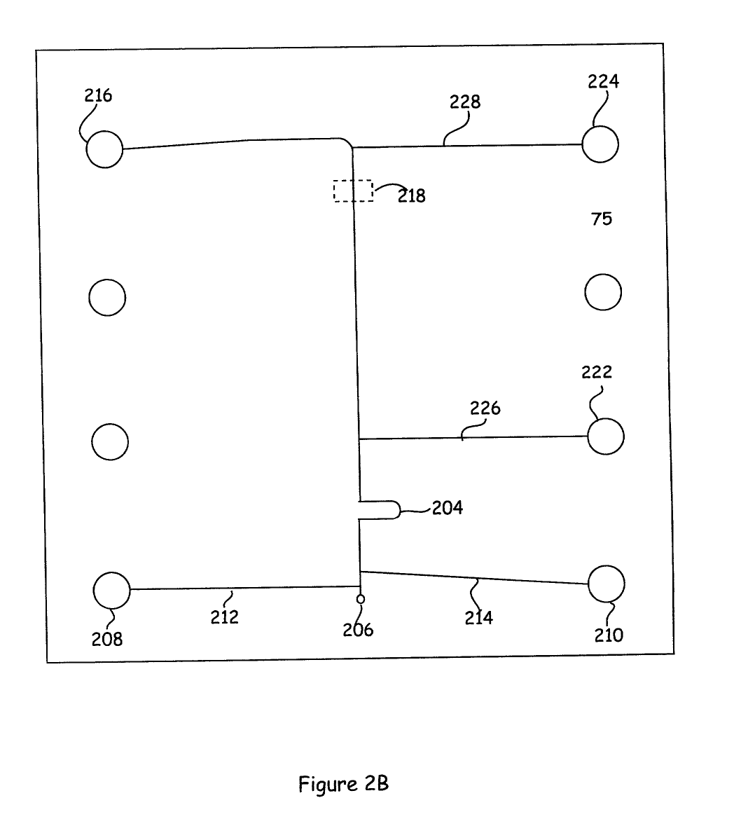

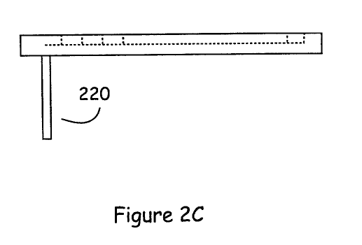

In particularly preferred aspects, the methods of the present invention are carried out in flowing microfluidic systems. In particular, the pooled target mixture, or component thereof, is flowed along a main reaction channel, while one or more test compounds are individually, serially or in a pool, introduced into the main channel to interact with the pooled target systems. The effects of the test compounds on the normal or control functioning of the pooled targets is then detected within the main channel at a point downstream from the point of mixture of all of the components. An example of microfluidic devices for carrying out such flowing assay methods is shown in FIG. 2A, 2B, and 2C.

FIG. 2A illustrates a microfluidic device channel pattern that is generally useful in carrying out fluorogenic assays. As shown, the overall device 200 includes a body structure 202. A main analysis channel 204 is provided disposed within an interior portion of the device. At one end of the reaction channel 204 is a capillary inlet 206 which forms the junction between the main analysis channel 204 and an external sampling capillary element (220, shown in the side view of FIG. 2C). Reagent reservoirs 208 and 210 are provided in the overall body structure, and are in fluid communication with the main reaction channel 204 via connecting channels 212 and 214, respectively. The main channel terminates at the end opposite the capillary inlet 206 at a waste reservoir 216.

In operation, the pooled target mixture is deposited into, e.g., reservoir 208. The target mixture is then flowed into the main reaction channel 204 through connecting channel 212. This is generally carried out by applying either a positive pressure to reservoir 208, or a negative pressure to waste reservoir 216, or a combination of the two, to control flow of material in a desired fashion. Test compounds are then drawn into the main reaction channel through capillary element 220 (FIG. 2C) and junction/inlet 206. Typically, multiple test compounds are introduced into the main channel in a serial fashion, one after the other. The test compounds then mix with the pooled target mixture. In most cases, other components of the target mixture are introduced into the assay reaction mixture after the test compounds have been introduced, in order to allow the test compound to interact with one component of the target systems before the additional components are added. For example, in the device shown in FIG. 2A, additional components of the pooled target system are deposited into reservoir 210 and are added to the reaction channel 204 after the test compounds have been added. Again, flowing of these additional components is generally accomplished by applying a positive pressure to reservoir 210, a negative pressure to waste reservoir 216, or a combination of the two. Typically, the latter case is preferred in that it provides the ability to accurately control flows from multiple reservoirs into common channels, simultaneously.

By way of example, in a cell-based pooled receptor target assay, a cell suspension that comprises different groups of cells bearing different receptor systems is deposited into reservoir 208. Meanwhile, a mixture of ligands or agonists to the pooled receptor target systems is deposited into reservoir 210. The receptor portion of the pooled system, e.g., the cell suspension, is flowed into the main reaction channel and mixed with test compounds brought in through the external sampling capillary. Ligands or agonists for the different receptors, are then brought into the main channel to mix with the receptor pool/test compound mixture.

The overall assay mixture is transported along the main reaction channel past a detection zone or window. The detection zone or window is typically defined as the region of the main analysis channel where a detector is in sensory communication with the contents of the reaction channel. In most cases, the detection zone or window is provided as a transparent region, in order to allow optical signals to be transmitted outside of the channel to a nearby or adjacent detector.

As noted, transporting the various reagents through the channels of the device is typically carried out by applying a pressure differential along the direction of desired fluid flow to push or draw fluids through the channels. This is typically accomplished by either applying differential positive pressures to each of the different reagent reservoirs, and/or applying a vacuum to the waste reservoir 216. Combinations of applied positive and negative pressures are typically used, in conjunction with tuned channels, e.g., having tuned flow resistances, to precisely control relative flows of reagents. Results of the assay are then monitored at a detection zone 218 along the main channel 204.

In non-fluorogenic assays, different channel geometries may be employed. For example, in performing an electrophoretic mobility shift screening assay, a device having the channel geometry shown in FIG. 2B is typically used (the device has the same side view profile shown in FIG. 2C). Common reference numerals are used for features that are common between different figures in this application, e.g., FIGS. 2A and 2B.

In operation, the various target system reagents are placed into reservoirs 208 and 212, as described for FIG. 2A above. Again, test compounds are drawn into the main reaction channel 204 through the external sampling capillary 220 (FIG. 2C) where they mix with the pooled target system reagents. As shown, the device shown in FIG. 2B includes two additional reservoirs 222 and 224 connected to different points along the main reaction channel, e.g., via channels 226 and 228, respectively. These reservoirs provide access ports for placing electrodes into the device. The electrodes are used to generate an electrical potential gradient along the main reaction channel. As the reaction mixture, including a test compound slug, passes into the portion of the main channel 204 between channels 226 and 228, those reagents are subjected to the applied electric field. When subjected to an applied electric field, the products and substrates, which differ in their level of charge, begin to electrophoretically separate. In the absence of any change in the overall rate of product generation, this electrophoretic separation is masked by the continuously flowing reagents in the system, e.g., a steady state of substrates and products exists throughout the portion of channel 204 between channels 226 and 228, yielding, e.g., a steady state fluorescent signal. However, where a test compound has an effect on the functioning of the target system, it results in a characteristic deviation in this steady state, and its accompanying signal. Specifically, where a product moves faster under the applied electric field, the existence of an inhibitor in the test compound slug results in a depletion of product in the space in advance of the test compound plug (because it has not filled in with product due to the inhibition of the reaction), and an accumulation of the slower moving substrate (as well as the following on product) either in or following the test compound plug. Such mobility shift assays are described in detail in, e.g., U.S. Pat. No. 5,942,443, and WO 99/64836, each of which is hereby incorporated herein by reference in its entirety for all purposes.

Although generally described in terms of introducing an already mixed target pool into the microfluidic device, e.g., depositing the pooled target system in reservoir 208, in the case of microfluidic systems, it is often desirable to pool target systems through the operation of the microfluidic system. Specifically, different target systems are provided in separate reservoirs that are each coupled to the main analysis channel, e.g., through one or different connecting channels. The device is then run in “pooled target” mode by simultaneously moving the different target system components from each of the target reservoirs, into the reaction channel. The screening assays are then carried out as described above. In the case where a test compound has an effect on the overall pooled target system, the screen can be readily repeated with each different target system, independently, by transporting each target system separately, e.g., not pooled, down the analysis channel while mixing in the test compound, to identify the target system that is affected. Thus, in place of reservoir 208 would be a plurality of reservoirs coupled to the main channel 204, where each reservoir contains a different target system.

In alternative embodiments, individual or discrete channel networks are used to screen each different test compound or groups of test compounds. In particular, the pooled target system is mixed with test compounds within a discrete reaction channel or a reservoir coupled to the channel. The reaction mixture is then transported through the channel to a detection point at which the results of the screen are detected/monitored. Such discrete assay channels are often used in cases where a screen assay is based upon, e.g., a mobility shift between substrates and products of the pooled target systems.

C. Detectors

A variety of detection systems are generally useful in accordance with the devices and systems of the present invention. Typically, such detectors are placed in sensory communication with the reaction vessels in which the screening assays are carried out, whether those vessels are fluidic channels, wells, or test tubes. As used herein, the phrase “in sensory communication” refers to a detector that is positioned such that it is capable of receiving a detectable signal from the contents of the reaction vessel that is being used for the screening assay. In the case of optical detectors, sensory communication typically requires that the detector be positioned adjacent to an open, or transparent or translucent portion of the reaction vessel such that an optical signal can be transmitted to and received by the detector from the contents of the reaction vessel. In the case of other detection systems, sensory communication can require that a sensor be in direct contact with the reaction vessel contents, e.g., placed in the reaction vessel or channel. Such detectors typically include, e.g., electrochemical sensors, i.e., pH or conductivity sensors, thermal sensors, and the like.

In preferred aspects, optical detectors are used to detect the signals from the screening reactions. In particular, as fluorescent signals are often employed in screening assays, fluorescence detection systems are most preferred. Typically, such systems employ a light source that is directed at the contents of the reaction vessel. Fluorescence emitted from the reaction vessel is then collected through an optical train and detected. As noted above, it is often desirable to utilize a detection system that is capable of distinguishing among signals from each of the different target systems in the pooled target mixture. In the case of fluorescent systems, such detection systems typically employ an optical train that is capable of separating and separately detecting fluorescent signals at different wavelengths. This typically involves the inclusion of different optical filters, dichroics and the like within the optical train. Examples of detection systems that are capable of distinguishing among a number of different fluorescent signals are described in, e.g., U.S. Pat. No. 5,821,058, which is generally described for use in performing nucleic acid, e.g., sequencing, detection.

As noted above, in some cases, fluorescence polarization detection is used to monitor a variety of assay results, e.g., binding or hybridization reactions, charge altering reactions, and the like. As such, in those cases, the detection system optionally employs fluorescence polarization detection. Such detection systems are described in, e.g., WO 99/64840, and WO 00/72016, which is incorporated herein by reference in its entirety for all purposes.

D. Control and Data Analysis

The systems of the present invention also typically include a processor operably connected to the detection system and, in the case of microfluidic embodiments, a flow controller, for storing and analyzing data received from the reaction vessel, and/or for directing the flow of material through the channels of the microfluidic channels of a microfluidic device. A variety of processors or computers are useful in conjunction with the present invention, including PC computers running Intel Pentium®, Pentium II®, or compatible CPUs, Apple MacIntosh® computers or the like.

V. Kits

The present invention also provides kits that are useful in practicing the methods described herein, without excessive set up, reagent preparation and the like, on the part of the user. The kits of the present invention typically include a reaction vessel, e.g., a multiwell plate or microfluidic device, as well as providing a plurality of target systems, either separately stored, or stored as a pooled target mixture. The kits also optionally include detectable dyes, buffers, and other reagents useful in carrying out the above-described methods. Finally, the kits of the invention typically include the various components packaged together along with instructions for carrying out those of the methods described herein that are desirable for a particular application.

VI. EXAMPLES

### Example 1

### Demonstrated Target Pooling in G-Protein Coupled Receptor System Assay

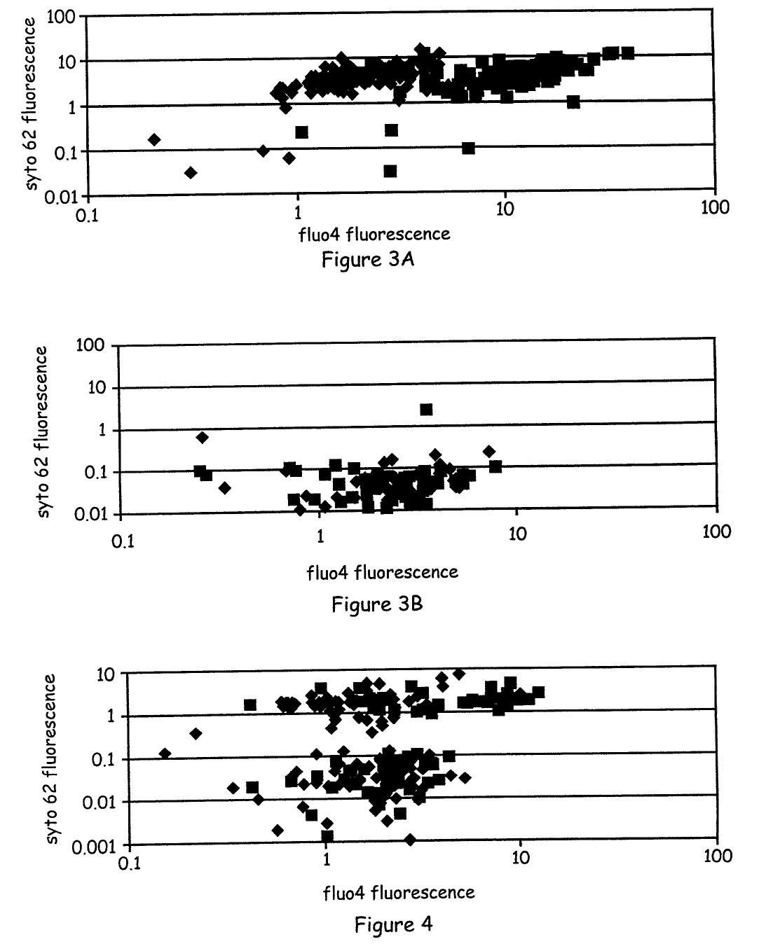

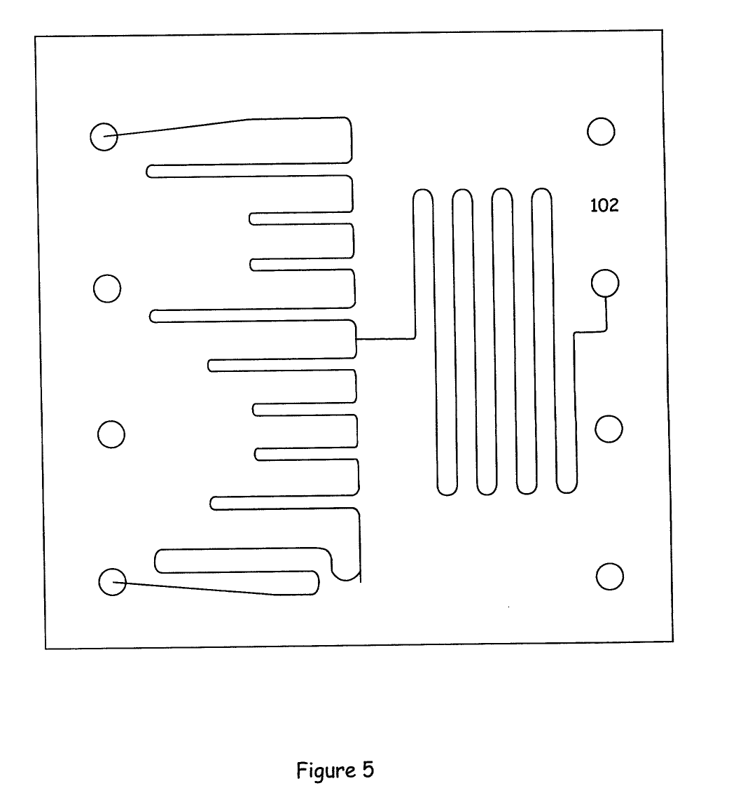

The present invention was demonstrated in a microfluidic format using two different cell lines in a single suspension as the pooled target system. Briefly, two Jurkat cell lines were provided in a single cell suspension, where one cell line was modified to express a G-coupled protein receptor, which could be activated by a known ligand. The cell suspension was introduced into a microfluidic device having the channel layout shown in FIG. 5. The two cell lines were distinguishable by differential labeling with SYTO 62, where the GPCR containing cell line had a relatively high level of SYTO 62 fluorescence, while the native cell was stained with a lower level of SYTO 62 fluorescence. Both cell lines were also stained with Fluo-4, an intracellular dye that indicates the presence of intracellular calcium ions. FIGS. 3A and 3B illustrate the fluorescent signals obtained when the two cell lines were run separately, e.g., in a non-pooled format, and exposed to the known GPCR ligand (1 μM—square, negative control—diamond). As can be seen, the introduction of the ligand causes an increase in Fluo-4 fluorescence (indicative of increased Calcium flux) only in the GPCR-expressing Jurkat cell lines. FIG. 4 shows the case where he target cell lines are pooled and run simultaneously in the same reaction channel. Again, the increase in Fluo-4 fluorescence is attributable to the subpopulation of cells having a higher level of SYTO-62 fluorescence, namely the GPCR containing cell line. Accordingly, it can be seen that two different cell lines were screened in an assay in one half of the time that it would have taken in the absence of the target pooling methods described herein.

### Example 2

### Two Target Screen for Dose Response to Different Stimuli

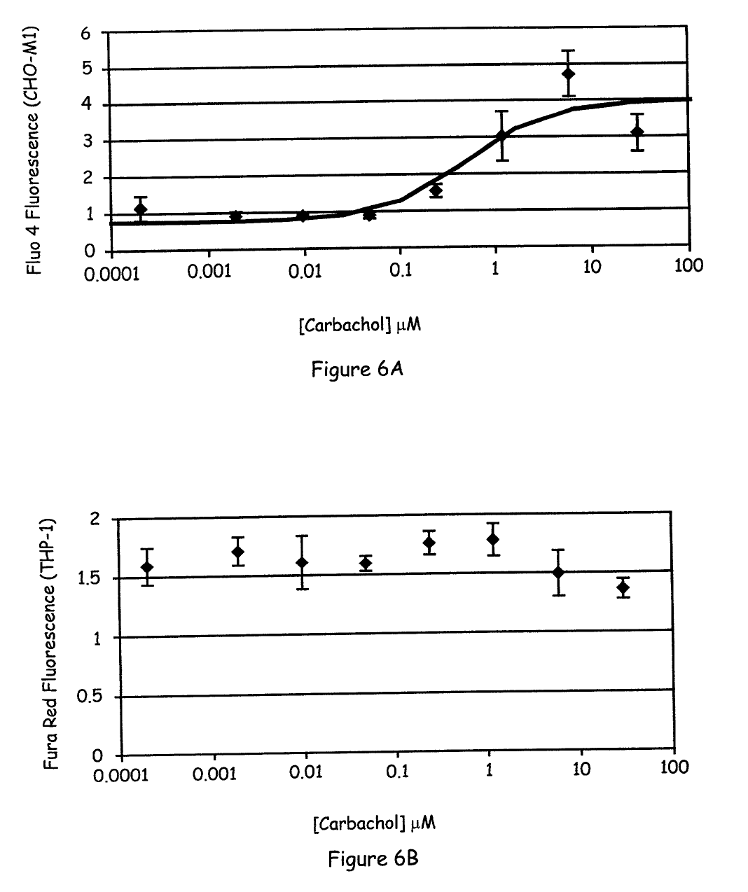

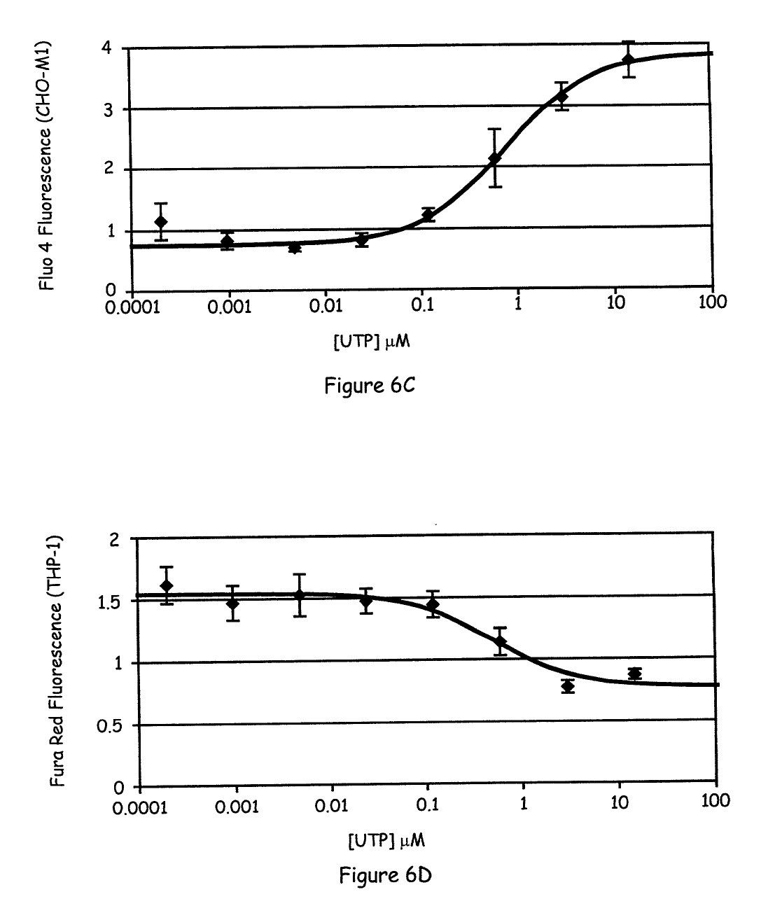

The invention was further demonstrated using two different cell lines in a single suspension as the pooled target system. Briefly, a CHO cell line expressing the M1 muscarinic receptor which is activated by carbachol, was labeled with Fluo-4. A THP-1 cell line was labeled with Fura red. Both cell lines were pooled and introduced into microfluidic device having the channel structure illustrated in FIG. 5. The cells were flowed through the main channel of the device past a detector that excited the cells with blue excitation light. Green fluorescence was analyzed from the Fluo-4 labeled CHO cells to measure their response to the test compounds. The measurement of the different fluorescence was accomplished simultaneously through the use of appropriate beam splitters and filters in the optical train of the detector. Similarly, red fluorescence was measured from the Fura-red labeled THP-1 cells to measure their response. Different concentrations of carbachol were flowed into contact with the cells and the green and red fluorescence was measured as an indication of each cell line's response, and the results were plotted (FIG. 6A-6B). Similarly, different concentrations of UTP were also contacted with the cells flowing through the channel and the results for each system were plotted (FIG. 6C-D). As can be seen from FIG. 6A-6B, carbachol causes an expected dose dependent change in the CHO-M1 cells bearing the Fluo-4 label, while FIG. 6C-6D illustrates that both cell lines exhibit a dose dependent response to UTP.

All publications and patent applications are herein incorporated by reference to the same extent as if each individual publication or patent application was specifically and individually indicated to be incorporated by reference. Although the present invention has been described in some detail by way of illustration and example for purposes of clarity and understanding, it will be apparent that certain changes and modifications may be practiced within the scope of the appended claims.

What is claimed is:

1. A method of performing a screening assay, comprising:
providing a first target mixture in a first reaction vessel, the first target mixture comprising at least first and second different target systems;
introducing at least a first test agent into the target mixture; and
determining an effect of the test agent on the first and second target systems.

2. The method of claim 1, wherein the first target system comprises at least a first component of a first biochemical system and at least a first component of a second biochemical system.

3. The method of claim 2, wherein the first target mixture comprises first and second interacting components of a first biochemical system, and the second target system comprises first and second interacting components of a second biochemical system.

4. The method of claim 1, wherein the first target system comprises at least three different target systems.

5. The method of claim 1, wherein the first target mixture comprises at least four different target systems.

6. The method of claim 1, wherein the first target mixture comprises at least six target systems.

7. The method of claim 1, wherein the first target mixture comprises at least ten target systems.

8. The method of claim 1, wherein the first target mixture comprises a first cell suspension, the first cell suspension comprising the first and second target systems.

9. The method of claim 8, wherein the first cell suspension is engineered to comprise the first and second target systems.

10. The method of claim 8, wherein the first cell suspension comprises at least first and second cell groups, the first cell group comprising the first target system and the second cell group comprising the second target system.

11. The method of claim 8, wherein the cell suspension comprises at least a first cell group that comprises both the first and second target systems.

12. The method of claim 1, wherein the first and second target systems produce first and second detectable signals, respectively, the first and second detectable signals being indicative of a function of the first and second target systems, respectively.

13. The method of claim 12, wherein the first detectable signal is distinguishable from the second detectable signal.

14. The method of claim 12, wherein the first and second detectable signals comprise first and second optically detectable signals.

15. The method of claim 14, wherein the first and second optically detectable signals comprise fluorescent signals.

16. The method of claim 15, wherein the first and second optically detectable signals comprise increases or decreases in a level of fluorescence.

17. The method of claim 15, wherein the first and second optically detectable signals comprise increases or decreases in a level of polarized fluorescence.

18. The method of claim 10, wherein the first and second target systems are independently receptor systems, signal transduction systems, ion channel systems, enzyme systems, hybridizing nucleic acid systems, nucleic acid-protein interacting systems, protein-protein interacting systems.

19. The method of claim 10, wherein at least one of the first and second target systems in the target mixture comprises a G-protein coupled receptor (GPCR) system.

20. The method of claim 19, wherein at least two of the at least first and second target systems in the first target mixture comprise GPCR systems.

21. The method of claim 10, wherein at least one of the first and second target systems in the first target mixture comprises a DNA-binding protein system.

22. The method of claim 1, wherein at least one of the first and second target systems in the first target mixture comprises a kinase enzyme system.

23. The method of claim 1, wherein at least one of the first and second target systems in the first target mixture comprises a phosphatase enzyme system.

24. The method of claim 1, wherein at least one of the first and second target systems in the first target mixture comprises a protease enzyme system.

25. The method of claim 12, wherein the target mixture comprises a first cell suspension, the first cell suspension comprising the first and second target systems and a reference signal, wherein the reference signal is substantially uniformly associated with all of the cells in the first cell suspension.

26. The method of claim 25, wherein the determining step comprises detecting the first or second signal, and the reference signal from cells in the cell suspension.

27. The method of claim 26, wherein the first or second signal and the reference signal is separately detected from individual cells in the first cell suspension.

28. The method of claim 25, wherein the reference signal comprises an optical signal.

29. The method of claim 28, wherein the reference signal comprises a fluorescent signal.

30. The method of claim 29, wherein the fluorescent signal is emitted from a fluorescent label that is substantially uniformly associated with all of the cells in the first suspension.

31. A system for performing high throughput screening assays, comprising:
a reaction vessel having a first target mixture disposed therein, the first target mixture comprising at least first and second target systems, the first target mixture being different from the first target system;
a test agent sampler for sampling a test agent and introducing the test agent into the reaction vessel; and
a detector positioned in sensory communication with the first target mixture, the detector being configured to detect an effect of a test agent on the first and second target systems.

32. The system of claim 31, wherein the target mixture comprises a liquid mixture.

33. The system of claim 31, wherein the target mixture comprises a particulate suspension.

34. The system of claim 33, wherein the target mixture comprises a first cell suspension, the first cell suspension comprising the at least first and second target mixtures.

35. The system of claim 31, wherein the reaction vessel is selected from a test tube, a cuvette, a well in a multiwell plate, and a fluid channel.

36. The system of claim 35, wherein the reaction vessel comprises a fluidic channel.

37. The system of claim 36, wherein the fluidic channel comprises at least one cross sectional dimension between about 0.1 and 500 μm.

38. The system of claim 36, wherein the fluidic channel is disposed in a planar substrate.

39. The system of claim 36, wherein the fluidic channel is intersected by at least a second fluidic channel.

40. The system of claim 39, wherein at least one of the first and second fluidic channels is fluidly connected to the test agent sampler.

41. The system of claim 31, further comprising a flow controller for flowing the first target mixture into and through the at least first fluidic channel.

42. The system of claim 31, wherein the detector comprises an optical detector, the optical detector being positioned adjacent to an open or transparent portion of the reaction vessel, such that the detector is capable of receiving an optical signal from the first target mixture.

43. The system of claim 42, wherein the detector comprises a fluorescent detector.
